(19) United States
(12) Patent Application Publication
(10) Pub. No.: US 2026/0337215 A1
(43) Pub. Date: Oct. 29, 2026

(54) COENZYME REGENERATION SYSTEM AND COENZYME REGENERATION METHOD

(71) Applicant: THE HONG KONG POLYTECHNIC UNIVERSITY, Hong Kong (CN)

(72) Inventors: Xuming ZHANG, Hong Kong (CN); Yao CHAI, Hong Kong (CN)

(21) Appl. No.: 19/703,703

(22) Filed: Jun. 10, 2026

Related U.S. Application Data
(63) Continuation of application No. PCT/CN2026/082820, filed on Mar. 11, 2026.

(30) Foreign Application Priority Data
Mar. 11, 2025 (CN) 202510284098.6

(51) Int. Cl. C07H 1/00; C07H 21/02

(52) U.S. Cl. CPC C07H 1/00; C07H 21/02

(57) ABSTRACT

This application discloses a coenzyme regeneration system and a coenzyme regeneration method. The coenzyme regeneration system includes: a metal sulfide, an oxidized coenzyme, and a solvent. The metal sulfide has a hexagonal phase crystal structure. The coenzyme regeneration system provided herein, by virtue of the hexagonal phase crystal structure of the metal sulfide, exhibits excellent light absorption characteristics and high electron-hole pair separation efficiency; this enables the coenzyme regeneration system, which utilizes the metal sulfide as a photocatalyst, to achieve coenzyme regeneration without relying on an electron mediator.

### CROSS-REFERENCE TO RELATED APPLICATIONS

This application is a continuation application of International Patent Application No. PCT/CN2026/082820 filed Mar. 11, 2026, and claims priority to the Chinese Patent Application No. 202510284098.6 filed Mar. 11, 2025, the entire contents of which are incorporated herein by reference.

### TECHNICAL FIELD

The present application relates to the field of photocatalytic technology, and specifically to a coenzyme regeneration system and a coenzyme regeneration method.

### BACKGROUND

The statements made herein provide background information related to the present application and do not necessarily constitute prior art.

Coenzymes such as NADH and NADPH are key participants in numerous redox reactions in biological systems and play an indispensable role in processes including energy metabolism and biosynthesis. Achieving their efficient regeneration is critical for driving biocatalytic reactions, reducing production costs, and expanding the application scope of biosynthesis.

Traditional coenzyme regeneration methods typically rely on electron mediators to facilitate electron and proton transfer. Represented by [Cp*Rh (bpy) H₂O]²⁺, these electron mediators can accept photogenerated electrons in photocatalytic systems and transfer them to the coenzyme NAD(P)⁺ while assisting proton transfer, thereby achieving coenzyme regeneration. However, these electron mediators have numerous drawbacks in practical applications. First, they suffer from poor stability; under photocatalytic reaction conditions, they are prone to decomposition and deactivation, leading to a gradual decline in catalytic efficiency and preventing sustained stable reaction performance. Second, the synthesis of these electron mediators is costly, thereby increasing the overall cost of the photocatalytic coenzyme regeneration process. Additionally, the effective recovery and reuse of these electron mediators remains challenging; this not only results in resource wastage and further increases costs but also poses potential environmental risks. These issues significantly limit the large-scale application and development of traditional photocatalytic coenzyme regeneration technologies. Accordingly, the development of novel, highly efficient, stable, and economically and environmentally friendly photocatalytic coenzyme regeneration systems remains a key unmet challenge in the field.

### SUMMARY

It is an objective of embodiments of the present application to provide a coenzyme regeneration system and a coenzyme regeneration method, including, but not limited to, addressing the problem of how to achieve photocatalytic coenzyme regeneration without relying on electron mediators.

The technical solutions adopted in the embodiments of the present application are as follows:

In a first aspect, a coenzyme regeneration system is provided. The coenzyme regeneration system comprises: a metal sulfide, an oxidized coenzyme, and a solvent. The metal sulfide has a hexagonal phase crystal structure.

In some embodiments, the metal sulfide comprises at least one of cadmium sulfide, tin sulfide, zinc sulfide, indium sulfide, and molybdenum sulfide.

In some embodiments, the metal sulfide is cadmium sulfide, and the cadmium sulfide has a feather-like morphology.

In some embodiments, the metal sulfide is cadmium sulfide, and a lattice spacing of a (100) crystal plane of the cadmium sulfide is 0.358 nm.

In some embodiments, the metal sulfide is cadmium sulfide, and an X-ray photoelectron spectrum of the cadmium sulfide exhibits characteristic peaks within ranges of 160 eV to 162 eV, 162 eV to 164 eV, 404 eV to 407 eV, and 410 eV to 414 eV.

In some embodiments, the metal sulfide is cadmium sulfide, and the cadmium sulfide comprises cadmium sulfide single crystals.

In some embodiments, the metal sulfide is tin sulfide, and the tin sulfide comprises tin sulfide nanosheets. The tin sulfide nanosheets have a thickness of 2 nm to 50 nm and a lateral dimension of 50 nm to 10μm.

In some embodiments, within the coenzyme regeneration system, the oxidized coenzyme has an initial concentration of 0.1 mM to 1 mM, and the metal sulfide has an initial concentration of 1.25 mg/mL to 25 mg/mL.

In some embodiments, the oxidized coenzyme comprises at least one of NAD⁺ and NADP⁺.

In some embodiments, the solvent comprises at least one of triethanolamine and deionized water.

In some embodiments, the coenzyme regeneration system further comprises a pH buffering agent, and the pH buffering agent comprises a phosphate-based buffering agent.

In some embodiments, the coenzyme regeneration system has a pH value of 7 to 8.5.

In a second aspect, a coenzyme regeneration method is provided. The coenzyme regeneration method comprises the following step:
subjecting the coenzyme regeneration system according to the first aspect to a photocatalytic reaction under irradiation of a visible light, thereby converting the oxidized coenzyme within the coenzyme regeneration system into a reduced coenzyme.

In an embodiment, the visible light has a wavelength of 400 nm to 720 nm.

In some embodiments, a vertical distance between the visible light source and the coenzyme regeneration system is 5 cm to 30 cm.

In some embodiments, the visible light source has a power of 50 W to 450 W; and/or, a duration of the photocatalytic reaction is 10 mins to 100 mins.

In some embodiments, the reduced coenzyme comprises at least one of 1,4-NADH and NADPH.

The advantages of the coenzyme regeneration system provided in embodiments of the present application are summarized as follows:

The coenzyme regeneration system according to the first aspect of the present application comprises a metal sulfide, an oxidized coenzyme, and a solvent. Having a hexagonal phase crystal structure, the metal sulfide exhibits excellent light absorption characteristics and high electron-hole pair separation efficiency. This enables the coenzyme regeneration system to convert oxidized coenzyme to reduced coenzyme without the need for an electron mediator, thereby regenerating the coenzyme.

The advantages of the coenzyme regeneration method provided in the embodiments of the present application are summarized as follows:

In the coenzyme regeneration method according to the second aspect of the present application, the coenzyme regeneration system described in the above embodiments utilizes the metal sulfide having excellent visible-light absorption capability and high electron-hole pair separation efficiency. This enables the metal sulfide to use an electron-proton coupling mechanism, thereby facilitating the efficient conversion of oxidized coenzymes to reduced coenzymes and achieving high conversion rates. As a result, this coenzyme regeneration system enables highly efficient photocatalytic coenzyme regeneration and has broad application prospects in the fields of energy conversion and biocatalysis.

### BRIEF DESCRIPTION OF THE DRAWINGS

To more clearly illustrate the technical solutions set forth in the embodiments of the present application, the accompanying drawings referred to in the following description of the embodiments or exemplary technical solutions are briefly introduced. It will be apparent that the drawings described below depict only some embodiments of the present application, and that other drawings may be obtained by those of ordinary skill in the art without inventive effort.

FIG. 1 is an X-ray diffraction analysis pattern of cadmium sulfide provided in Examples 1-5 of the present application;

FIG. 2 is a full-scan X-ray photoelectron spectrum of cadmium sulfide provided in Examples 1, 4, and 5 of the present application;

FIG. 3 is an X-ray photoelectron analysis pattern of cadmium sulfide provided in Examples 1, 4, and 5 of the present application;

FIG. 4 is a transmission electron microscopy image of cadmium sulfide provided in Example 4 of the present application;

FIG. 5 is a selected area electron diffraction analysis pattern of cadmium sulfide provided in Example 4 of the present application;

FIG. 6 is an energy-dispersive X-ray spectrum of cadmium sulfide provided in Example 4 of the present application;

FIG. 7 is an X-ray diffraction analysis pattern of tin sulfide provided in Examples 6 and 7 of the present application;

FIG. 8 is a 1H NMR spectrum of the product obtained from the photocatalytic reduction of NAD⁺;

FIG. 9 is an illustration of the coenzyme regeneration performance of cadmium sulfide provided in the examples of the present application;

FIG. 10 is an illustration of the coenzyme regeneration performance of tin sulfide provided in the examples of the present application;

FIG. 11 is an analysis of the photocatalytic performance of cadmium sulfide in catalyzing NADH regeneration at different wavelengths;

FIG. 12 is a reaction kinetics analysis of the photocatalytic regeneration of NADH by cadmium sulfide;

FIG. 13 is a stability test of the photocatalytic regeneration of NADH by cadmium sulfide;

FIG. 14 is a UV-Vis absorption spectrum of the reaction solution for the photocatalytic regeneration of NADPH using cadmium sulfide, provided in Example 4 of the present application;

FIG. 15 is a UV-Vis absorption spectrum of the reaction solution for the photocatalytic regeneration of NADPH using cadmium sulfide, provided in Example 1 of the present application;

FIG. 16 is a schematic diagram illustrating the mechanism of photocatalyst-catalyzed NADH regeneration provided in the present application;

FIG. 17 is a Gibbs free energy diagram for the photocatalytic regeneration of NADH using cadmium sulfide, provided in Example 4 of the present application.

### DETAILED DESCRIPTION OF THE EMBODIMENTS

To render the objectives, technical solutions, and advantages of the present application clearer and more comprehensible, the present application is described in further detail below with reference to the accompanying drawings and embodiments. It should be understood that the specific embodiments described herein are provided solely to explain the present application and are not intended to limit the scope of the present application.

It should be noted that when a component is referred to as being “fixed to” or “disposed on” another component, it may be directly on the other component or indirectly on the other component. When a component is referred to as being “connected to” another component, it may be directly or indirectly connected to the other component. The directional or positional relationships indicated by terms such as “upper,” “lower,” “left,” “right,” etc., are based on the directional or positional relationships shown in the accompanying drawings; these terms are used solely for the convenience of description and are not intended to indicate or imply that the device or element referred to must have a specific orientation, or be constructed and operated in a specific orientation. Therefore, these terms should not be construed as limiting the scope of the present application; for a person of ordinary skill in the art, the specific meanings of the aforementioned terms may be interpreted according to the specific context. The terms “first,” “second,” etc., are used solely for descriptive purposes and should not be construed as indicating or implying relative importance, nor as implicitly specifying the quantity of technical features. The term “multiple” signifies two or more, unless explicitly and specifically defined otherwise.

The weights of the relevant components mentioned in the description of the embodiments of the present application refer not only to the specific content of each component but may also denote the proportional weight relationships among the components; therefore, any formulation in which the content of the relevant components is proportionally scaled up or down relative to the embodiments disclosed herein falls within the scope of the present application. Specifically, the masses described in the description of the embodiments of the present application may be expressed in units of mass well-known in the field of chemical engineering, such as μg, mg, g, kg, and the like.

The term “NADH” refers to reduced nicotinamide adenine dinucleotide; the term “NADPH” refers to reduced nicotinamide adenine dinucleotide phosphate.

A first aspect of the embodiments of the present application provides a coenzyme regeneration system, comprising: a metal sulfide, an oxidized coenzyme, and a solvent; and the metal sulfide has a hexagonal phase crystal structure.

The coenzyme regeneration system provided in the embodiments of the present application comprises a metal sulfide, an oxidized coenzyme, and a solvent. Since the metal sulfide has a hexagonal phase crystal structure, it contributes, on the one hand, to the formation of a relatively narrow band gap, enabling the metal sulfide to absorb light across a broad range of wavelengths, particularly visible light. Consequently, when the metal sulfide is irradiated with visible light, the photon energy facilitates the excitation of valence-band electrons to the conduction band, generating photogenerated carriers; thus, the metal sulfide exhibits excellent light absorption characteristics. On the other hand, the hexagonal phase crystal structure is characterized by non-centrosymmetry, meaning that the centers of positive and negative charges within the crystal structure do not coincide. This characteristic facilitates the formation of a built-in electric field within the crystal, which exerts opposing forces on electrons and holes, driving them to migrate in opposite directions and thereby effectively separating the electron-hole pairs. In addition, the hexagonal phase crystal exhibits anisotropy; this anisotropy enables electrons and holes to migrate rapidly along distinct pathways, thereby achieving the effective separation of electron-hole pairs.

By virtue of their favorable light absorption characteristics and superior electron-hole pair separation capabilities, metal sulfides are able to effectively capture light energy, thereby facilitating highly efficient energy transfer. Subsequently, by harnessing the electron-proton coupling effect, they significantly accelerate the conversion of oxidized coenzymes into their reduced forms, enabling the coenzyme regeneration system to achieve regeneration without relying on external electron mediators.

In some embodiments, the metal sulfide comprises at least one of cadmium sulfide, tin sulfide, zinc sulfide, indium sulfide, and molybdenum sulfide.

These metal sulfides exhibit excellent visible-light absorption capabilities and highly efficient electron-hole separation properties; consequently, by virtue of an electron-proton coupling mechanism, the coenzyme regeneration system is enabled to achieve photocatalytic coenzyme regeneration without relying on electron mediators.

In some embodiments, the metal sulfide is cadmium sulfide, and the cadmium sulfide has a feather-like morphology.

Feather-like cadmium sulfide has a large specific surface area, thereby providing a greater number of active sites for the generation and transfer of photogenerated charges. Consequently, during photocatalytic reactions, these abundant active sites facilitate the more rapid reaction of photogenerated electrons and photogenerated holes with the substrate, thereby significantly enhancing the separation efficiency of the photogenerated charges. This enables a coenzyme regeneration system containing cadmium sulfide to achieve photocatalytic coenzyme regeneration without relying on electron mediators.

In some embodiments, the metal sulfide is cadmium sulfide, and a lattice spacing of a (100) crystal plane of the cadmium sulfide is 0.358 nm.

The lattice spacing of the (100) crystal plane of cadmium sulfide falling within such range indicates that the cadmium sulfide has a hexagonal phase crystal structure, thereby endowing the cadmium sulfide with high photogenerated charge separation efficiency.

In some embodiments, the metal sulfide is cadmium sulfide, and an X-ray photoelectron spectrum of the cadmium sulfide exhibits characteristic peaks within ranges of 160 eV to 162 eV, 162 eV to 164 eV, 404 eV to 407 eV, and 410 eV to 414 eV.

The characteristic peaks observed in the X-ray photoelectron spectrum of the cadmium sulfide in the range of 160 eV to 162 eV and the range of 162 eV to 164 eV correspond to the 2p orbital characteristic peaks of sulfur, such as S 2p_{3/2} and S 2p_{1/2}, indicating that the valence state of sulfur in the cadmium sulfide is −2. The characteristic peaks within the ranges of 404 eV to 407 eV and 410 eV to 414 eV correspond to the Cd 3d_{5/2} peak of cadmium, indicating that the valence state of cadmium in the cadmium sulfide is +2.

In some embodiments, the metal sulfide is cadmium sulfide, and the cadmium sulfide comprises cadmium sulfide single crystals.

Because cadmium sulfide single crystals have fewer grain boundary defects, the risk of carrier trapping at grain boundaries is effectively reduced, thereby significantly enhancing the migration efficiency of photogenerated electron-hole pairs. Furthermore, the electron migration pathways within the cadmium sulfide single crystals are more continuous, allowing photogenerated carriers to be transported to catalytic sites more efficiently, which is very beneficial for enhancing quantum efficiency. Meanwhile, cadmium sulfide single crystals exhibit high stability and resistance to photocorrosion, reducing the likelihood of reactions such as “CdS+O₂→CdSO₄”. This endows cadmium sulfide single crystals with broad application prospects in the field of photocatalytic coenzyme regeneration.

In some embodiments, the metal sulfide is tin sulfide, and the tin sulfide comprises tin sulfide nanosheets. The tin sulfide nanosheets have a thickness of 2 nm to 50 nm and a lateral dimension of 50 nm to 10 μm.

Exemplarily, the thickness of the tin sulfide nanosheets may be 2 nm, 5 nm, 8 nm, 10 nm, 15 nm, 20 nm, 25 nm, 30 nm, 35 nm, 40 nm, 45 nm, 50 nm, or other typical and non-limiting values.

Lateral dimension refers to the maximum extent to which the tin sulfide nanosheet extends in the lateral direction. Exemplarily, the lateral dimension of the tin sulfide may be 50 nm, 80 nm, 100 nm, 500 nm, 1 μm, 2 μm, 3 μm, 4 μm, 5 μm, 6 μm, 7 μm, 8 μm, 9 μm, 10 μm, or other typical and non-limiting values.

The tin sulfide nanosheet has a suitable band structure, enabling the tin sulfide nanosheet to effectively absorb visible light; this absorption triggers the excitation of electrons, causing them to transition from the valence band to the conduction band and thereby generating photogenerated electron-hole pairs. In particular, the tin sulfide nanosheet features a large specific surface area, which facilitates the exposure of a greater number of active sites. Consequently, when visible light illuminates the surface of the tin sulfide nanosheet, a larger quantity of photogenerated electron-hole pairs can be produced. In addition, the tin sulfide nanosheet exhibits an appropriate thickness, which ensures that the photogenerated charge carriers traverse only a short migration distance; this effectively shortens the charge diffusion pathway and reduces the probability of internal recombination between electron-hole pairs. On this basis, the tin sulfide nanosheet demonstrates excellent light absorption characteristics and high electron-hole pair separation capability, thereby enabling tin sulfide nanosheet to achieve photocatalytic coenzyme regeneration without relying on external electron mediators.

In some embodiments, tin sulfide comprises tin sulfide single crystals.

Tin sulfide single crystals exhibit a low electron-hole recombination rate, which facilitates the efficient separation of photogenerated carriers and significantly enhances photocatalytic efficiency. Furthermore, tin sulfide single crystals have high structural stability, making it easier to maintain excellent catalytic activity and stability during long-term use; consequently, tin sulfide single crystals have broad prospects for application in the field of photocatalytic coenzyme regeneration.

In some embodiments, within the coenzyme regeneration system, the oxidized coenzyme has an initial concentration of 0.1 mM to 1 mM, and the metal sulfide has an initial concentration of 1.25 mg/mL to 25 mg/mL.

Exemplarily, the initial concentration of the oxidized coenzyme may be 0.1 mM, 0.2 mM, 0.3 mM, 0.4 mM, 0.5 mM, 0.6 mM, 0.7 mM, 0.8 mM, 0.9 mM, 1 mM, or other typical and non-limiting values; and the initial concentration of the metal sulfide may be 1.25 mg/mL, 2 mg/mL, 5 mg/mL, 7.5 mg/mL, 10 mg/mL, 15 mg/mL, 20 mg/mL, 22 mg/mL, 25 mg/mL, or other typical and non-limiting values.

Within this range, the metal sulfide is enabled to fully exert its photocatalytic activity, providing appropriate active sites and accelerating the reaction rate of the oxidized coenzyme.

In some embodiments, within the coenzyme regeneration system, the initial concentration of the oxidized coenzyme is 0.25 mM to 0.75 mM, and the concentration of the metal sulfide is 5 mg/mL to 10 mg/mL.

Exemplarily, the initial concentration of the oxidized coenzyme may be 0.25 mM, 0.375 mM, 0.5 mM, 0.625 mM, 0.75 mM,, or other typical and non-limiting values. The initial concentration of the metal sulfide may be 5 mg/mL, 6 mg/mL, 7.5 mg/mL, 9 mg/mL, 10 mg/mL, or other typical and non-limiting values.

Within this range, an appropriate initial concentration of the oxidized coenzyme ensures that the coenzyme regeneration system contains sufficient reactants to participate in electron transfer, thereby maintaining a dynamic equilibrium between the oxidized and reduced states of the coenzyme and, in turn, enhancing reaction efficiency. Moreover, a suitable initial concentration of the metal sulfide provides an adequate number of active sites to efficiently catalyze the reaction; this not only improves the coenzyme conversion efficiency but also enhances economic viability, thereby helping to prevent resource waste and minimize cost increases.

In some embodiments, the oxidized coenzyme comprises at least one of NAD⁺ and NADP⁺.

Due to the favorable light absorption property and high electron-hole pair separation efficiency of the metal sulfide, the coenzyme regeneration system containing the metal sulfide is capable of achieving coenzyme regeneration without relying on electron mediators.

In some embodiments, the solvent comprises at least one of triethanolamine and deionized water.

Under the conditions of this solvent system, the components are facilitated to mix thoroughly, allowing the oxidized coenzyme to come into close contact with the metal sulfide, thereby greatly improving the electron transfer efficiency and accelerating the reaction process of coenzyme regeneration.

In some embodiments, the solvent comprises an aqueous solution of triethanolamine having a weight-to-volume concentration of 10-30% (w/v).

In some embodiments, the coenzyme regeneration system further comprises a pH buffering agent, and the pH buffering agent comprises a phosphate-based buffering agent.

The phosphate-based buffering agent is capable of maintaining the pH value of the coenzyme regeneration system within a relatively stable range through its inherent acid-base buffering action, thereby providing a suitable conversion environment for the oxidized coenzyme and facilitating the efficient progress of the coenzyme regeneration reaction.

Exemplarily, the phosphate-based buffering agent includes, but is not limited to, at least one of phosphoric acid, dihydrogen phosphates, and hydrogen phosphates. Dihydrogen phosphates include, but are not limited to, at least one of sodium dihydrogen phosphate, potassium dihydrogen phosphate, and ammonium dihydrogen phosphate; and hydrogen phosphates include, but are not limited to, at least one of sodium hydrogen phosphate, potassium hydrogen phosphate, and ammonium hydrogen phosphate.

In some embodiments, the coenzyme regeneration system has a pH value of 7 to 8.5.

This pH range renders the change in Gibbs free energy of the coenzyme reaction favorable for the generation of the reduced coenzyme, which helps drive the conversion of the oxidized coenzyme toward the reduced form, thereby enhancing the efficiency and yield of coenzyme regeneration.

A second aspect of embodiments of the present application provides a coenzyme regeneration method, comprising the following step:
subjecting the coenzyme regeneration system provided in the first aspect to a photocatalytic reaction under irradiation of a visible light, thereby converting the oxidized coenzyme within the coenzyme regeneration system into a reduced coenzyme.

The coenzyme regeneration method provided in the second aspect of embodiments of the present application utilizes a coenzyme regeneration system as described in the aforementioned embodiments that incorporates the metal sulfide having excellent visible-light absorption capability and high electron-hole pair separation efficiency. This enables these metal sulfides to leverage an electron-proton coupling mechanism, thereby facilitating the highly efficient conversion of oxidized coenzymes into reduced coenzymes and achieving high conversion rates. Precisely because this coenzyme regeneration system enables the efficient photocatalytic regeneration of coenzymes, it holds broad prospects for application in the fields of energy conversion and biocatalysis.

The coenzyme regeneration system provided in the embodiments of the present application enables the efficient regeneration of coenzymes through electron-proton coupling, without relying on external electron mediators. By dispensing with the need for any electron mediators, this coenzyme regeneration system simplifies the overall photocatalytic reaction setup, thereby reducing operational complexity. This simplification facilitates the industrial application of the method, particularly in large-scale catalytic and energy conversion processes, while lowering costs and enhancing the operational feasibility of the reaction.

Furthermore, the regeneration process of the coenzyme regeneration system provided in the present application overcomes the issues associated with traditional methods, specifically, the instability of electron mediators, the difficulty of their recovery, and their high cost. This renders the coenzyme regeneration reaction process more streamlined and efficient, offering broad prospects for application, particularly in the fields of biocatalysis and energy conversion.

In some embodiments, the step of subjecting the coenzyme regeneration system provided in the first aspect to a photocatalytic reaction under irradiation of a visible light is specifically performed as follows:
the coenzyme regeneration system is stirred in the dark for 10 mins to 60 mins, and the photocatalytic reaction is carried out under visible light irradiation.

By stirring in the dark, the various components within the coenzyme regeneration system, such as oxidized coenzymes and metal sulfides are thoroughly mixed. This establishes a solid foundation of uniform material distribution for the subsequent photocatalytic reaction, thereby enabling the reaction to proceed more evenly and efficiently.

In some embodiments, the visible light has a wavelength of 400 nm to 720 nm. Exemplarily, the wavelength of the visible light may 400 nm, 420 nm, 450 nm, 475 nm, 500 nm, 600 nm, 720 nm, or other typical and non-limiting values.

In some embodiments, the wavelength of the visible light is 400 nm to 480 nm.

Metal sulfides exhibit excellent light absorption properties within the visible light region of 400 nm to 720 nm; they can effectively induce electron transitions within the metal sulfides, generating photogenerated electrons and photogenerated holes. This process initiates photocatalytic reactions, thereby enabling the metal sulfides to exert their optimal catalytic activity and promoting the regeneration of reduced coenzymes.

The coenzyme regeneration system disclosed herein is capable of driving the conversion of NAD⁺ and NADP⁺ to NADH and NADPH under visible light irradiation. This not only enhances the simplicity and environmental friendliness of photocatalytic coenzyme regeneration but also circumvents issues such as poor stability and difficulty in recovery that may arise with electron mediators during the catalytic process, thereby reducing reaction costs and bolstering the sustainability of photocatalytic reactions.

In some embodiments, a vertical distance between the visible light source and the coenzyme regeneration system is 5 cm to 30 cm. Exemplarily, the vertical distance may be 5 cm, 10 cm, 15 cm, 20 cm, 25 cm, 30 cm, or other typical and non-limiting values.

Within this distance range, the coenzyme regeneration system is able to receive light of appropriate intensity, thereby enabling the metal sulfide to fully absorb light energy to drive the coenzyme regeneration reaction.

In some embodiments, the visible light source has a power of 50 W to 450 W.

Within this power range, sufficient light energy can be provided for the photocatalytic reaction of metal sulfides to generate an adequate number of photogenerated electrons and photogenerated holes, thereby effectively driving the regeneration of reduced coenzymes and enhancing both the reaction rate and coenzyme regeneration efficiency.

In some embodiments, the duration of the photocatalytic reaction is 10 mins to 100 mins.

Within this time range, the metal sulfide in the coenzyme regeneration system is afforded sufficient time to be excited by visible light, generating photogenerated electrons and photogenerated holes; this facilitates the participation of these charge carriers in the coenzyme regeneration process, thereby promoting coenzyme regeneration and achieving a high regeneration conversion rate.

In some embodiments, the reduced coenzyme comprises at least one of 1,4-NADH and NADPH.

With respect to the oxidized coenzyme, not only is a high regeneration efficiency achieved, but excellent selectivity is also demonstrated, for instance, the selectivity for 1,4-NADH can reach 100%. This implies that, in relevant reactions, metal sulfides are capable of highly specifically promoting the regeneration of 1,4-NADH, thereby significantly enhancing the purity and efficiency of the photocatalytic reaction.

In addition to coenzyme NADH, coenzyme NADPH is another vital coenzyme that plays an extensive role in cellular energy metabolism and biosynthetic reactions. Within the cell, NADPH primarily functions as a reducing agent, providing the essential reducing power required for the synthesis of fatty acids, nucleotides, and amino acids; and also serves to neutralize reactive oxygen species within the cellular antioxidant defense system. Consequently, the regeneration of NADPH is of profound significance for maintaining normal cellular metabolism and physiological functions. Much like the regeneration mechanism of NADH, the regeneration of NADPH is accomplished through the acceptance of protons and electrons by NADP⁺.

In some embodiments, the metal sulfide used in the coenzyme regeneration system and the coenzyme regeneration method may be prepared using the following method, which includes the following steps:

In step S10, a metal source and a sulfur source are subjected to a hydrothermal reaction within a solution system to obtain the metal sulfide; in which, the metal source comprises at least one of a cadmium source, a tin source, a zinc source, an indium source, and a molybdenum source.

In some embodiments, in step S10, the sulfur source comprises at least one of thiourea, thioacetamide, and L-cystine.

In some embodiments, in step S10, the metal source comprises a soluble salt corresponding to a metal element.

The soluble salt corresponding to the metal element includes, but is not limited to, at least one of chlorides, nitrates, or acetates.

Exemplarily, the cadmium source includes, but is not limited to, at least one of cadmium chloride (CdCl₂·2.5H₂O), cadmium nitrate (Cd(NO₃)₂·4H₂O), or cadmium acetate ((CH₃COO)₂Cd·2H₂O).

Exemplarily, the tin source comprises, but is not limited to, at least one of tin(IV) acetate (SnCl₄·5H₂O) and tin(IV) chloride (Sn(CH₃CO₂)₄).

In some embodiments, the molar ratio of the metal element in the metal source to the sulfur element in the sulfur source is less than or equal to a stoichiometric ratio of the metal element to the sulfur element in the metal sulfide; that is, the sulfur element is present in relative excess.

Exemplarily, in the preparation of cadmium sulfide, a molar ratio of cadmium from the cadmium source to sulfur from the sulfur source is 1:(1-5).

Exemplarily, in the preparation of tin sulfide, a molar ratio of tin from the tin source to sulfur from the sulfur source is 1:(2-5).

In some embodiments, in step S10, the solvent employed in the solution system comprises at least one of deionized water, ethylene glycol, and triethanolamine.

In some embodiments, in step S10, the solvent employed in the solution system comprises at least one of ethylene glycol and triethanolamine.

In some embodiments, in step S10, the solvent employed in the solution system comprises deionized water and ethylene glycol; in which, a volume ratio of deionized water to ethylene glycol may be 1:3, 3:1, 1:1, or other typical and non-limiting values.

This is because the solvent exerts a certain influence on the surface chemical composition, defect states, and surface charge characteristics of the metal sulfide.

Taking water and ethylene glycol as examples, their distinct characteristics lead to vastly different interactions with the metal sulfide. Water is highly polar, having a dielectric constant ε of approximately 80. This high polarity gives rise to two critical issues when water is employed as the solvent for the synthesis of the metal sulfide. First, the surface of the metal sulfide is highly prone to adsorbing hydroxyl groups, resulting in the formation of metal-hydroxyl complexes. Second, certain metal sulfides which are susceptible to partial hydrolysis in an aqueous medium, such as tin sulfide, face a risk of hydrolysis. Such occurrences compromise the photocatalytic properties of the metal sulfide. Furthermore, some metal sulfides that are sensitive to oxidation, such as cadmium sulfide, are prone to photo-oxidation in the aqueous phase, thereby undermining their overall stability. In contrast, ethylene glycol is a weakly polar, highly viscous solvent with a dielectric constant of approximately 37. On one hand, the hydroxyl groups within the ethylene glycol molecules can coordinate with the metal ions in the metal sulfide to form stable metal-solvent complexes; this effectively mitigates surface hydroxylation and, consequently, enhances the stability of the catalyst. On the other hand, due to its lower polarity, ethylene glycol can significantly suppress the extent of metal sulfide hydrolysis, thereby preserving the structural integrity of the metal sulfides. Moreover, ethylene glycol exhibits low oxygen solubility, which serves to prevent the photo-oxidation of metal sulfides and further bolsters their stability.

In aqueous systems, the surface defect states of metal sulfides are subject to multifaceted influences. First, the interaction between water and the metal sulfide surface carries the risk of partially dissolving the metal sulfide, which may consequently promote the formation of sulfur (S) defects on the surface. Second, metal sulfides are prone to oxidation in water, and the resulting oxidation products are highly likely to diminish photocatalytic activity. Furthermore, defect centers formed on the metal sulfide surface within an aqueous phase may evolve into electron-hole recombination centers; this shortens the lifetime of photogenerated charge carriers and exerts a detrimental effect on catalytic efficiency. In contrast, within an ethylene glycol environment, ethylene glycol exerts a protective effect on the surface structure of the metal sulfide, effectively reducing the concentration of sulfur vacancies and thereby enhancing the stability of the catalyst. Due to its lower oxygen solubility, ethylene glycol is able to inhibit the formation of oxides and suppress surface oxidation reactions, thereby significantly extending the photocatalytic lifetime of the catalyst. Moreover, ethylene glycol serves to prevent the excessive hydrolysis of metal sulfides, thereby preserving their catalytic activity.

Regarding the surface charge of metal sulfides, the high polarity of water tends to induce a charge screening effect, which may compromise the transport efficiency of photogenerated electrons within the metal sulfides. Ethylene glycol, having a lower dielectric constant, can effectively reduce the electron-hole recombination rate and thereby enhance photocatalytic efficiency.

Specifically, when metal sulfides are synthesized in an aqueous phase, their surfaces are prone to hydroxylation, that is, the surface of the metal sulfide readily adsorbs hydroxyl groups. This phenomenon may interfere with the electron-proton transfer process, consequently diminishing the coenzyme regeneration rate to a certain extent. Furthermore, coenzymes in an aqueous phase are susceptible to photocatalytic oxidation; this is particularly problematic when the metal sulfides generate excessive reactive oxygen species (such as O₂⁻ and ¹O₂), as it can lead to a reduction in coenzyme regeneration efficiency. In contrast, within an organic environment, organic solvents serve to stabilize the surface structure of the metal sulfides, thereby anchoring the catalytic sites more firmly, bolstering the sustainability of the photocatalytic cycle, and ultimately achieving an enhancement in coenzyme regeneration efficiency. Additionally, organic solvents can suppress the formation of the reactive oxygen species O₂⁻, thereby minimizing the degradation of coenzymes through side reactions and improving overall selectivity.

Based on this, the present application enables the effective regulation of the catalytic activity of metal sulfides through the judicious selection of solvents. For instance, in scenarios where an aqueous phase is unfavorable for enhancing catalytic activity, a suitable organic solvent is selected to construct a mixed-solvent system in conjunction with the aqueous phase. This mixed-solvent system allows for the full utilization of the organic solvent's inherent properties, such as stabilizing the surface structure of the metal sulfides, minimizing the generation of surface defects, inhibiting the formation of reactive oxygen species, and reducing oxidative damage to coenzymes. Through these synergistic effects, the system not only enhances the sustainability of the photocatalytic cycle, thereby enabling the metal sulfides to maintain high efficiency over prolonged reaction periods, but also substantially boosts the catalytic activity of the metal sulfides, consequently leading to a significant improvement in coenzyme regeneration efficiency.

In some embodiments, in step S10, a mass ratio of a combined mass of the metal source and the sulfur source to a mass of the solvent is 1:(90 to 100). Exemplarily, the mass ratio of the combined mass of the metal source and the sulfur source to the mass of the solvent may be 1:90, 1:92, 1:95, 1:98, 1:100, or other typical and non-limiting values.

Within this range, the raw materials are facilitated to mix fully and uniformly; furthermore, the reaction rate and efficiency are enhanced, and the occurrence of side reactions is minimized. Consequently, the purity and the yield of the target product are improved, while the stability and the reproducibility of the preparation process are ensured.

In some embodiments, in step S10, the conditions for the hydrothermal reaction include: a temperature of 100 °C to 200 °C and a duration of 6 hrs to 24 hrs.

Exemplarily, the temperature of the hydrothermal reaction may be 100 °C, 110 °C, 120 °C, 130 °C, 140 °C, 150 °C, 160 °C, 170 °C, 180 °C, 190 °C, 200 °C, or other typical and non-limiting values.

Exemplarily, the duration of the hydrothermal reaction may be 6 hrs, 8 hrs, 10 hrs, 12 hrs, 16 hrs, 18 hrs, 20 hrs, 24 hrs, or other typical and non-limiting values.

The hydrothermal reaction conditions falling within this range can facilitate the complete reaction of the metal and sulfur elements, thereby yielding the metal sulfide having a hexagonal crystalline structure.

The following description is provided in conjunction with specific embodiments.

### Example 1

This example provides a cadmium sulfide and a preparation method thereof.

The provided cadmium sulfide (CdS) has a hexagonal phase crystal structure.

The cadmium sulfide was prepared by the following steps:

In step 1, 1 mmol of cadmium chloride (CdCl₂·2.5H₂O) and 1.5 mmol of thiourea were added into a solvent (40 mL of deionized water) and were fully dissolved. A resulting mixed solution was then transferred into a 50 mL polytetrafluoroethylene (PTFE)-lined high-pressure autoclave, and was then sealed in a stainless steel vessel for a hydrothermal reaction, which was performed at a reaction temperature of 180 °C for a reaction time of 24 hrs.

In step 2, after the hydrothermal reaction was completed, the product was filtered, washed with deionized water, and then dried in a vacuum oven at 60 °C, and the cadmium sulfide having a hexagonal phase crystal structure was obtained.

### Example 2

This example provides a cadmium sulfide and a preparation method thereof. This example is the same as Example 1 except that the solvent was ethylene glycol and deionized water in a volume ratio of 1:3. Specifically, the solvent included 10 mL of ethylene glycol and 30 mL of deionized water.

### Example 3

This example provides a cadmium sulfide and a preparation method thereof. This example is the same as Example 1 except that the solvent was ethylene glycol and deionized water in a volume ratio of 1:1. Specifically, the solvent included 20 mL of ethylene glycol and 20 mL of deionized water.

### Example 4

This example provides a cadmium sulfide and a preparation method thereof. This example is the same as Example 1 except that the solvent was ethylene glycol and deionized water in a volume ratio of 3:1. Specifically, the solvent included 30 mL of ethylene glycol and 10 mL of deionized water.

### Example 5

This example provides a cadmium sulfide and a preparation method thereof. This example is the same as Example 1 except that the solvent was 40 mL of ethylene glycol.

### Example 6

This example provides a tin sulfide and a preparation method thereof.

The provided tin sulfide (SnS₂) has a hexagonal phase crystal structure.

The tin sulfide was prepared by the following steps:

In step 1, 1 mmol of tin tetrachloride (SnCl₄·5H₂O) and 2 mmol of thiourea were added to a solvent (40 mL of ethylene glycol) and were fully dissolved. A resulting mixed solution was then transferred into a 50 mL polytetrafluoroethylene (PTFE)-lined high-pressure autoclave, which was then sealed in a stainless steel vessel for a hydrothermal reaction, which was performed at a reaction temperature of 180 °C for a reaction time of 24 hrs.

In step 2, after the hydrothermal reaction was completed, the product was filtered, washed with deionized water, and then dried in a vacuum oven at 60 °C, and tin sulfide having a hexagonal phase crystal structure was obtained.

### Example 7

This example provides a tin sulfide and a preparation method thereof. This example is the same as Example 6 except that the solvent was 40 mL of deionized water.

### Microstructure and Surface Properties—Cadmium Sulfide

### (1) X-Ray Diffraction Analysis

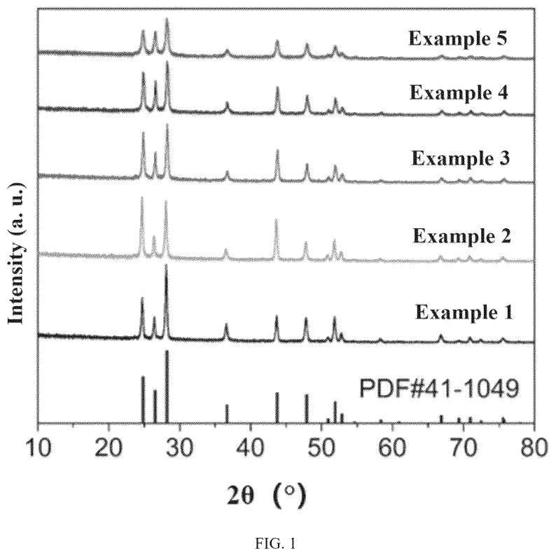

The metal sulfides prepared in Examples 1-5 were subjected to X-ray diffraction (XRD) analysis, and the results are shown in FIG. 1. As was clearly evident from FIG. 1, the diffraction peaks of the metal sulfide CdS prepared in Examples 1-5 were closely aligned with the standard diffraction peaks of hexagonal phase CdS (JCPDS No. 41-1049). Furthermore, no impurity peaks were observed, indicating that the metal sulfides prepared in Examples 1-5 of the present application exhibited a typical hexagonal phase crystal structure, possessed high purity, and were free from other impurity components.

### (2) X-Ray Photoelectron Spectroscopy

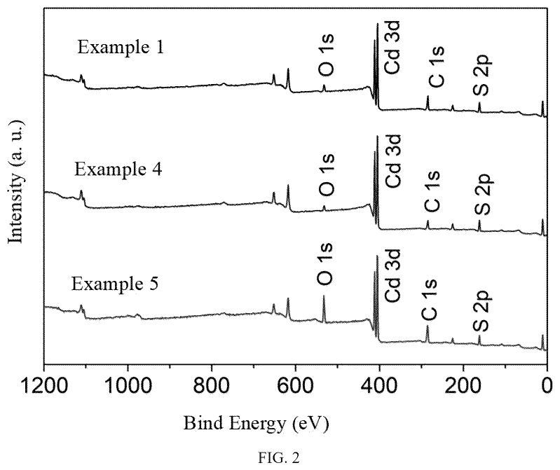

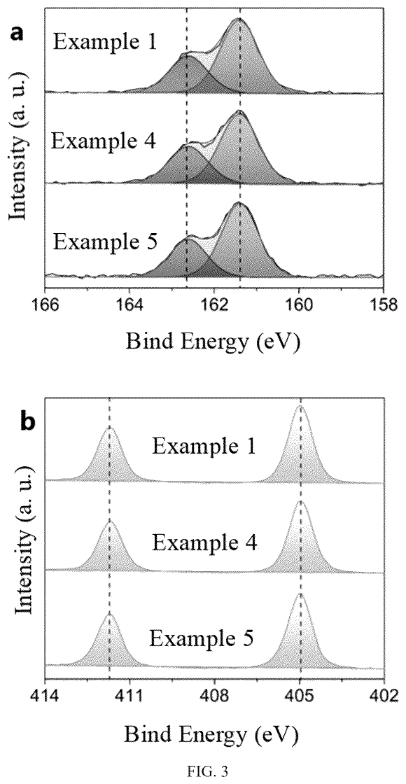

X-ray photoelectron spectroscopy (XPS) was employed to analyze the elemental composition and valence states of the metal sulfide CdS prepared in Examples 1 and 4-5, and the results are shown in FIGS. 2-3. As was clearly evident from FIG. 2, the full XPS spectrum revealed the presence of Cd, S, C, and O, indicating that the metal sulfides all contained the elements Cd and S. Part a of FIG. 3 showed that the metal sulfide prepared in Example 4 exhibited characteristic peaks at binding energies of 161.43 eV and 162.63 eV; these corresponded to the S 2p orbital characteristic peaks, specifically S 2p_{3/2} and S 2p_{1/2}, of the metal sulfide CdS, with a spin-orbit splitting component Δ of 1.20 eV. Part b of FIG. 3 showed that the metal sulfide prepared in Example 4 exhibited characteristic peaks at binding energies of 404.98 eV and 411.71 eV; these corresponded to the Cd 3d_{5/2} and Cd 3d_{3/2} orbitals of the metal sulfide CdS, with a spin-orbit splitting component Δ of 6.73 eV, corresponding to the Cd²⁺ state. Furthermore, the S spectra of the metal sulfides prepared in Examples 1 and 5 were found to be consistent with the CdS spectrum obtained in Example 4, indicating that the chemical environments and valence states of the metal sulfides prepared in Examples 1, 4, and 5 were similar.

### (3) Transmission Electron Microscopy

The metal sulfide prepared in Example 4 was analyzed using transmission electron microscopy (TEM), and the results are shown in FIG. 4. As can be seen from FIG. 4, the metal sulfide CdS exhibited a typical feather-like morphology, with a lattice spacing of 0.358 nm being observed for its (100) crystal plane.

### (4) Selected Area Electron Diffraction Analysis

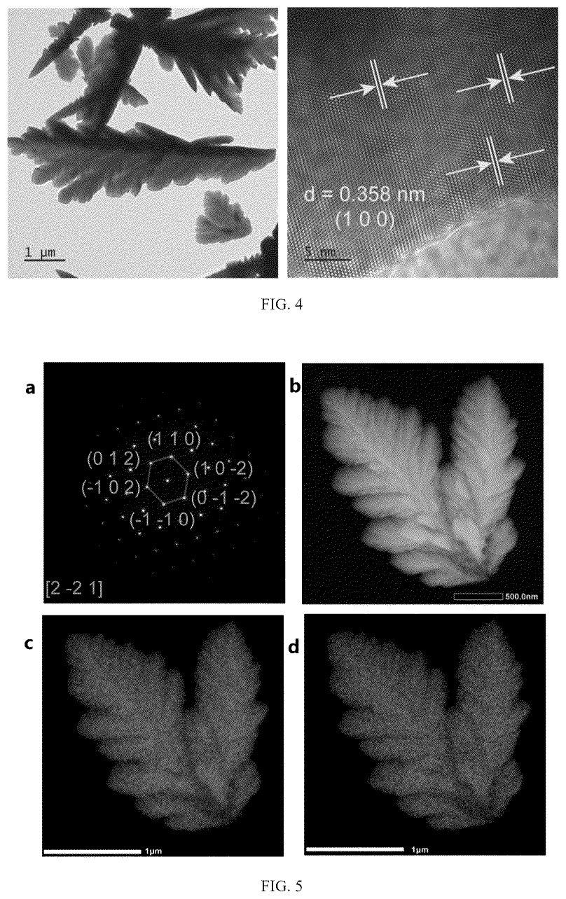

The metal sulfide prepared in Example 4 was subjected to selected area electron diffraction (SAED) analysis, and the results are shown in FIG. 5. Part a of FIG. 5 revealed that the metal sulfide CdS exhibited diffraction spots consistent with a single-crystal structure. The mapping analyses presented in part a, part b, and part c of FIG. 5 demonstrated that the elements S and Cd were uniformly distributed across surface of the nanofeather-like metal sulfide CdS.

### (5) Energy-Dispersive X-Ray Spectroscopy

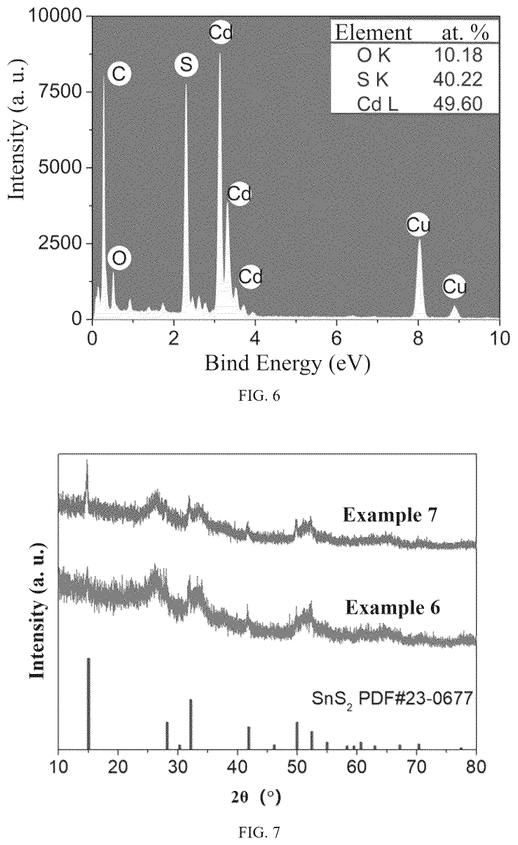

Energy-dispersive X-ray spectroscopy (EDS) was employed to confirm the elemental composition and stoichiometry of the metal sulfides. The EDS spectrum of the metal sulfide prepared in Example 4 is shown in FIG. 6. FIG. 6 clearly revealed the presence of Cd and S elements, with the Cd/S atomic ratio being observed to closely approach 1:1. These characterizations indicated that the successful preparation of CdS having a single-crystal structure was achieved.

### Microstructure and Surface Properties—Tin Sulfide

### (1) X-Ray Diffraction Analysis

The metal sulfides prepared in Examples 6-7 were subjected to X-ray diffraction (XRD) analysis, and the results are shown in FIG. 7. As was clearly evident from FIG. 7, the diffraction peaks of the metal sulfide SnS₂ prepared in Examples 6-7 were closely aligned with the standard diffraction peaks of hexagonal phase SnS₂ (JCPDS No. 23-0677). Furthermore, no impurity peaks were observed, indicating that the metal sulfides prepared in Examples 6-7 of the present application exhibited a typical hexagonal phase crystal structure, possessed high purity, and were free from other impurity components.

### Coenzyme Regeneration System and Coenzyme Regeneration Method

### Example S1

This example provides a coenzyme regeneration system comprising a metal sulfide (cadmium sulfide), an oxidized coenzyme (NAD⁺), a pH buffering agent, and a solvent (triethanolamine and deionized water). In this system, the concentration of the metal sulfide is 7.5 mg/mL; the concentration of the oxidized coenzyme is 0.25 mM; the pH buffering agent consists of sodium dihydrogen phosphate and disodium hydrogen phosphate; and the pH of the coenzyme regeneration system ranges from 7.8 to 8.0.

The specific procedure for preparing this coenzyme regeneration system was as follows:

A 30 mL quartz test tube was used as the reactor for the coenzyme regeneration system. 30 mg of the metal sulfide, 1 mL of an aqueous NAD⁺ solution (1 mM), 1 mL of a TEOA solution (an aqueous solution of triethanolamine, 15.0% w/v), and 2 mL of a buffer solution (an aqueous solution of the pH buffering agent, pH=7.4) were added to the reactor to obtain the coenzyme regeneration system.

A coenzyme regeneration method was performed by the following steps:

After stirring the coenzyme regeneration system in the dark for 30 mins, a xenon lamp was switched on to initiate photocatalysis, yielding a reaction dispersion. A 1 mL aliquot of the reaction dispersion was sampled every 30 mins. A 1 mL syringe equipped with a filter membrane was used to filter out the catalyst from the 1 mL dispersion aliquot, thereby obtaining a clear reaction solution. A PerkinElmer UV-Vis-NIR spectrophotometer was used to detect the NADH present in the reaction solution. The scanning range of the PerkinElmer UV-Vis-NIR spectrophotometer was 250 nm to 800 nm, and the scanning speed was 8 nm·s⁻¹. The yield of NADH was determined using the external standard curve method.

A 300 W xenon lamp equipped with a 420 nm cutoff filter was used to simulate the visible light radiation spectrum of sunlight; the distance between the quartz reactor (containing the coenzyme regeneration system) and the xenon lamp was set at 15 cm. After stirring the aforementioned coenzyme regeneration system in the dark for 30 mins, the xenon lamp was switched on to initiate photocatalysis, yielding a reaction dispersion. A 1 mL aliquot of the reaction dispersion was sampled every 30 mins. A 1 mL syringe equipped with a filter membrane was used to filter out the catalyst from the 1 mL dispersion aliquot, thereby obtaining a clear reaction solution. A PerkinElmer UV-Vis-NIR spectrophotometer was used to detect the NADH present in the reaction solution. The scanning range of the PerkinElmer UV-Vis-NIR spectrophotometer was 250 nm to 800 nm, and the scanning speed was 8 nm·s⁻¹. The yield of NADH was determined using the external standard curve method.

### Example S2

This example provides a coenzyme regeneration system and a coenzyme regeneration method. This example is the same as Example S1 except that the metal sulfide in the reactor was replaced with the CdS prepared in Example 2.

### Example S3

This example provides a coenzyme regeneration system and a coenzyme regeneration method. This example is the same as Example S1 except that the metal sulfide in the reactor was replaced with the CdS prepared in Example 3.

### Example S4

This example provides a coenzyme regeneration system and a coenzyme regeneration method. This example is the same as Example S1 except that the metal sulfide in the reactor was replaced with the CdS prepared in Example 4.

### Example S5

This example provides a coenzyme regeneration system and a coenzyme regeneration method. This example is the same as Example S1 except that the metal sulfide in the reactor was replaced with the CdS prepared in Example 5.

### Example S6

This example provides a coenzyme regeneration system and a coenzyme regeneration method. This example is the same as Example S4 except that the 420 nm cutoff filter was replaced with a monochromatic filter having a wavelength of 420 nm.

### Example S7

This example provides a coenzyme regeneration system and a coenzyme regeneration method. This example is the same as Example S4 except that the 420 nm cutoff filter was replaced with a monochromatic filter having a wavelength of 450 nm.

### Example S8

This example provides a coenzyme regeneration system and a coenzyme regeneration method. This example is the same as Example S4 except that the 420 nm cutoff filter was replaced with a monochromatic filter having a wavelength of 475 nm.

### Example S9

This example provides a coenzyme regeneration system and a coenzyme regeneration method. This example is the same as Example S4 except that the concentration of NAD⁺ in the coenzyme regeneration system was 0.75 mM.

### Example S10

This example provides a coenzyme regeneration system and a coenzyme regeneration method. This example is the same as Example S4 except that the concentration of NAD⁺ in the coenzyme regeneration system was0.625 mM.

### Example S11

This example provides a coenzyme regeneration system and a coenzyme regeneration method. This example is the same as Example S4 except that the concentration of NAD⁺ in the coenzyme regeneration system was 0.5 mM.

### Example S12

This example provides a coenzyme regeneration system and a coenzyme regeneration method. This example is the same as Example S4 except that the concentration of NAD⁺ in the coenzyme regeneration system was 0.375 mM.

### Example S13

This example provides a coenzyme regeneration system and a coenzyme regeneration method. This example is the same as Example S4 except that a cycling experiment was conducted, with the specific procedure as follows:

After stirring the coenzyme regeneration system in the dark for 30 mins, a xenon lamp was turned on to initiate the photocatalytic reaction; the reaction proceeded for 60 mins, yielding a Reaction Dispersion-1.

The Reaction Dispersion-1 was centrifuged and filtered, followed by the addition of 1 mL of NAD⁺ solution (having a concentration of 1 mM), 1 mL of TEOA solution (aqueous triethanolamine solution having a concentration of 15.0 w/v%), and 2 mL of buffer solution (pH=7.4), thereby forming a Coenzyme Regeneration System-2. After stirring the Coenzyme Regeneration System-2 in the dark for 30 mins, the xenon lamp was turned on to initiate photocatalysis; the reaction proceeded for 60 mins, yielding a Reaction Dispersion-2.

The Reaction Dispersion-2 was centrifuged and filtered, followed by the addition of 1 mL of NAD⁺ solution (having a concentration of 1 mM), 1 mL of TEOA solution (aqueous triethanolamine solution having a concentration of 15.0 w/v%), and 2 mL of buffer solution (pH=7.4), thereby forming a Coenzyme Regeneration System-3. After stirring the Coenzyme Regeneration System-3 in the dark for 30 mins, the xenon lamp was turned on to initiate photocatalysis; the reaction proceeded for 60 mins, yielding a Reaction Dispersion-3.

The Reaction Dispersion-3 was centrifuged and filtered, followed by the addition of 1 mL of NAD⁺ solution (having a concentration of 1 mM), 1 mL of TEOA solution (aqueous triethanolamine solution having a concentration of 15.0 w/v%), and 2 mL of buffer solution (pH=7.4), thereby forming a Coenzyme Regeneration System-4. After stirring the Coenzyme Regeneration System-4 in the dark for 30 mins, the xenon lamp was turned on to initiate photocatalysis; the reaction proceeded for 60 mins, yielding a Reaction Dispersion-4.

The Reaction Dispersion-5 was centrifuged and filtered, followed by the addition of 1 mL of NAD⁺ solution (having a concentration of 1 mM), 1 mL of TEOA solution (aqueous triethanolamine solution having a concentration of 15.0 w/v%), and 2 mL of buffer solution (pH=7.4), thereby forming a Coenzyme Regeneration System-5. After stirring the Coenzyme Regeneration System-5 in the dark for 30 mins, a xenon lamp was switched on to initiate photocatalysis; the reaction was allowed to proceed for 60 mins, yielding a Reaction Dispersion-5.

Using a 1 mL syringe equipped with a filter membrane, an aliquot of the reaction mixture was drawn from each of the Reaction Dispersion-1, the Reaction Dispersion-2, the Reaction Dispersion-3, the Reaction Dispersion-4, and the Reaction Dispersion-5, respectively. Upon completion of sampling, the syringe plunger was depressed to pass the reaction mixture through the filter membrane, thereby obtaining a clear reaction solution. A PerkinElmer UV-Vis-NIR spectrophotometer was used to detect the NADH present in each reaction solution. The scanning range of the PerkinElmer UV-Vis-NIR spectrophotometer was 250 nm to 800 nm, and the scanning speed was 8 nm·s⁻¹. The yield of NADH was determined using the external standard curve method.

### Example S14

This example provides a coenzyme regeneration system and a coenzyme regeneration method. This example is the same as Example S1 except that NAD⁺ in the reactor was replaced with NADP⁺, which has a concentration of 0.25 mM.

### Example S15

This example provides a coenzyme regeneration system and a coenzyme regeneration method. This example is the same as Example S4 except that NAD⁺ in the reactor was replaced with NADP⁺, which has a concentration of 0.25 mM.

### Example S16

This example provides a coenzyme regeneration system and a coenzyme regeneration method. This example is the same as Example S1 except that the metal sulfide in the reactor was replaced with SnS₂ prepared in Example 7.

### Comparative Example

### Comparative Example DS1

This comparative example provides a coenzyme regeneration system and a coenzyme regeneration method. This comparative example is the same as Example S4 except that 50 μL of an electron mediator solution ([Cp*Rh(bpy)H₂O]² solution) was added to the coenzyme regeneration system.

### Comparative Example DS2

This comparative example provides a coenzyme regeneration system and a coenzyme regeneration method. This comparative example is the same as Example S4 except that NAD⁺ was not added to the coenzyme regeneration system.

### Comparative Example DS3

This comparative example provides a coenzyme regeneration system and a coenzyme regeneration method. This comparative example is the same as Example S1 except that 50 μL of an electron mediator solution ([Cp*Rh(bpy)H₂O]² solution) was added to the coenzyme regeneration system, and NAD⁺ was replaced with NADP⁺ (at a concentration of 0.25 mM).

### Comparative Example DS4

This comparative example provides a coenzyme regeneration system and a coenzyme regeneration method. This comparative example is the same as Example S4 except that 50 μL of an electron mediator solution ([Cp*Rh(bpy)H₂O]² solution) is added to the coenzyme regeneration system, and NAD⁺ is replaced with NADP⁺ (at a concentration of 0.25 mM).

### (1) Selectivity Testing

The catalytically regenerated coenzyme NADH was analyzed. Specifically, the reaction solution prepared in Example S4 was characterized using ¹H NMR spectroscopy (Jeol ECZ500R spectrometer), utilizing deuterated water as the reference standard. The results are presented in FIG. 8. As evident from FIG. 8, the chemical shifts of the regioselective hydrogen atoms for 1,2-NADH, 1,4-NADH, and 1,6-NADH were observed at 6.63 ppm, 6.85 ppm, and 7.01 ppm, respectively. The peak at 6.85 ppm corresponded to 1,4-NADH; this indicates that, in the absence of an electron mediator, the reduction product of NAD⁺ is the physiologically active 1,4-NADH. No signals corresponding to 1,6-NADH or 1,2-NADH were detected, thereby demonstrating that the hexagonal phase crystalline CdS photocatalyst prepared in the present application exhibits a 100% selectivity toward 1,4-NADH.

### (2) Electron Mediator Dependence Test

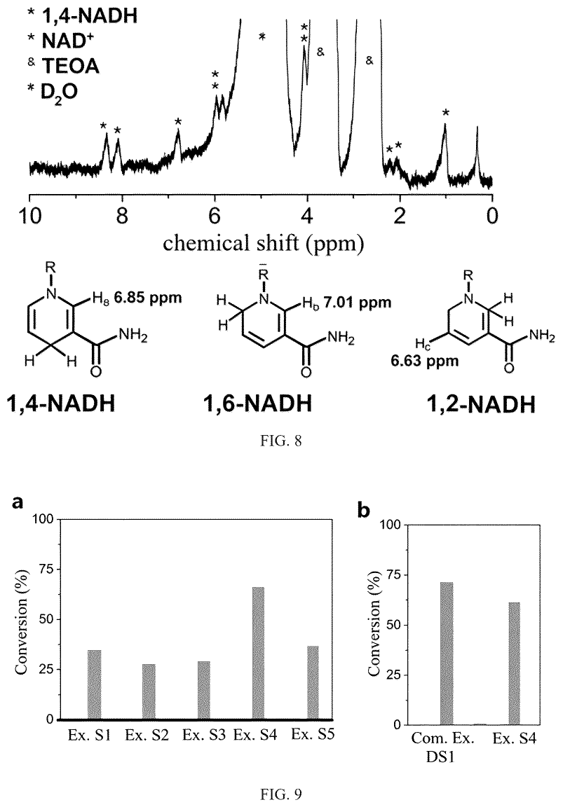

The results of the coenzyme regeneration methods described in Examples S1-S5 are shown in part a of FIG. 9, while the results of the photocatalytic coenzyme regeneration in Example S4 and Comparative Example DS1 are shown in part b of FIG. 9.

As can be seen from part a of FIG. 9, the NADH regeneration activities of the coenzyme regeneration systems provided in Examples S1-S5 all follow a typical “volcano-type” catalytic reaction pattern; notably, the CdS photocatalyst provided in Example S4 exhibits the highest NADH regeneration activity.

As can be seen from part b of FIG. 9, under conditions where an electron mediator was added, the NAD⁺ conversion rate reached 72.7% (at 1 hr). Surprisingly, however, even in the absence of an electron mediator, the photocatalyst provided in Example S4 was still able to catalyze the conversion of NAD⁺ to NADH, achieving a conversion rate of 66.0% (at 1 hr). This indicates that, even without the addition of an electron mediator, a coenzyme regeneration system containing metal sulfides can still demonstrate high coenzyme conversion efficiency, an efficiency that is, in fact, comparable to the level achieved when an electron mediator is present.

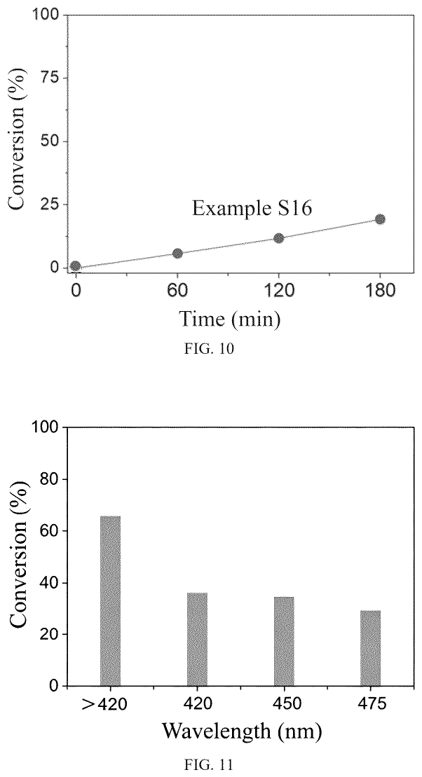

The results of the coenzyme regeneration method described in Example S16 are shown in FIG. 10. As can be seen from the figure, for the SnS₂ nanosheet photocatalyst provided in Example 7, the NAD⁺ conversion rate reached 19.3% (at 3 hrs) in the absence of an electron mediator. Based on this, it can be concluded that tin sulfide nanosheets are capable of achieving photocatalytic coenzyme regeneration without relying on an electron mediator.

### (3) Effect of Visible Light Wavelength

The results of photocatalytic coenzyme regeneration for Examples S4 and S6-S8 are presented in FIG. 11. As is clearly evident from FIG. 11, under monochromatic light irradiation, the catalytic activity of the metal sulfides decreases as the wavelength of the visible light increases; for instance, the conversion rate of NAD+ was 36.51% (after 1 h) at 420 nm, whereas it was 29.72% (after 1 h) at 475 nm.

### (4) Reaction Kinetics

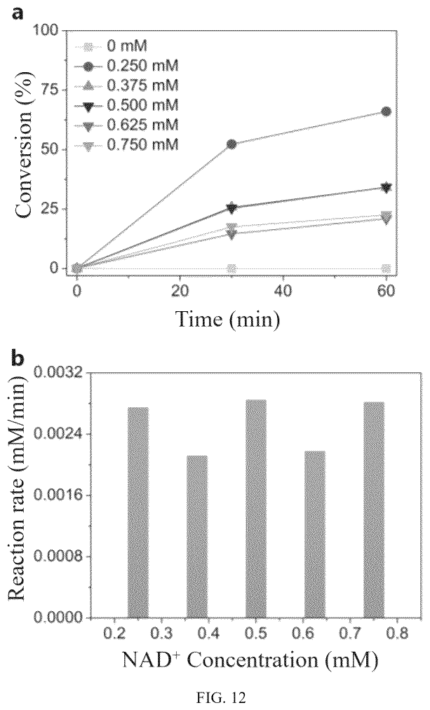

The results of the photocatalytic coenzyme regeneration experiments for Examples S4, S9-S12, and Comparative Example DS2 are presented in FIG. 12. Specifically, the time-conversion plots for the photocatalysts are shown in part a of FIG. 12, while the average reaction rates are shown in part b of FIG. 12.

The average reaction rate is defined as the ratio of the amount of product generated during a specific time interval of the photocatalytic reaction to the duration of that interval, specifically, the ratio of the yield of the coenzyme NADH to the reaction time elapsed.

As can be observed from FIG. 12(a), within a certain concentration range, the conversion rate of the substrate exhibits a downward trend as the concentration of the reaction substrate (NAD⁺) increases. For instance, when the initial concentration of NAD⁺ is 0.75 mM, the conversion rate after one hour is merely 22.59%; conversely, when the initial concentration of NAD⁺ is 0.25 mM, the conversion rate after one hour reaches 63.91%. FIG. 12(b) demonstrates that as the initial concentration of NAD⁺ increases, the chemical reaction rate remains essentially constant, indicating that the reaction follows zero-order kinetics. This phenomenon is primarily attributed to the fact that the reaction rate is limited by certain factors independent of reactant concentrations, such as the recombination rate of electron-hole pairs.

### (5) Cycling Performance

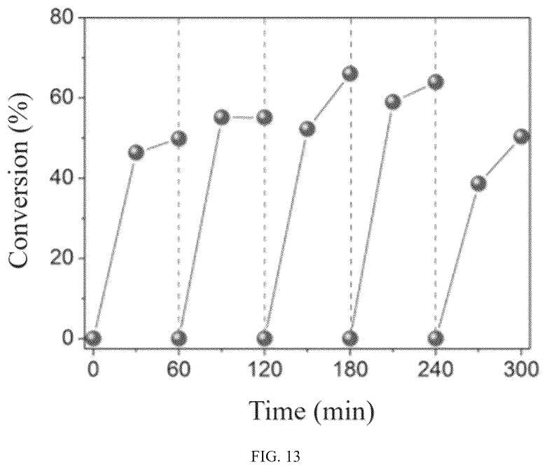

The results of the cycling performance test for the coenzyme regeneration system are shown in FIG. 13. As can be seen from the figure, after 300 mins of cycling, the photocatalytic activity of the CdS provided in Example 4 did not exhibit any significant decline, demonstrating excellent cycling stability.

### (6) UV-Vis Analysis

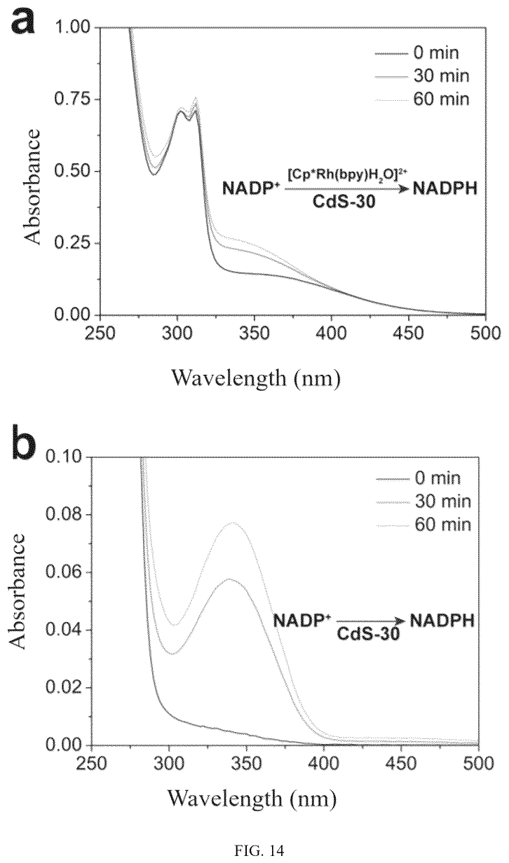

The UV-Vis absorption spectra of the reaction solutions provided in Example S15 and Example DS4 were measured. The results are shown in FIG. 14.

Part a of FIG. 14 presents the UV-Vis absorption spectrum of the reaction solution from Example DS4; this spectrum was recorded in the presence of the electron mediator [Cp*Rh(bpy)H₂O]²⁺. A distinct UV-Vis absorption peak was observed at 340 nm, which is characteristic of NADPH. As the irradiation time increased, the absorption peak intensified, indicating the generation of NADPH. From 0 min to 60 mins, the absorption intensity of NADPH increased by 0.1136. Part b of FIG. 14 presents the UV-Vis absorption spectrum of the reaction solution from Example S15; this spectrum was recorded without the addition of the electron mediator [Cp*Rh(bpy)H₂O]²⁺. At an irradiation time of 0 min, no distinct absorption peak was detected at 340 nm. However, as the irradiation time extended to 30 mins and 60 mins, a clear UV-Vis absorption peak emerged at 340 nm, and its intensity increased over time. From 0 min to 60 mins, the absorption intensity of NADPH rose by 0.0723. This demonstrates that the CdS photocatalyst provided in Example 4 is capable of achieving NADPH regeneration even without the addition of an electron mediator.

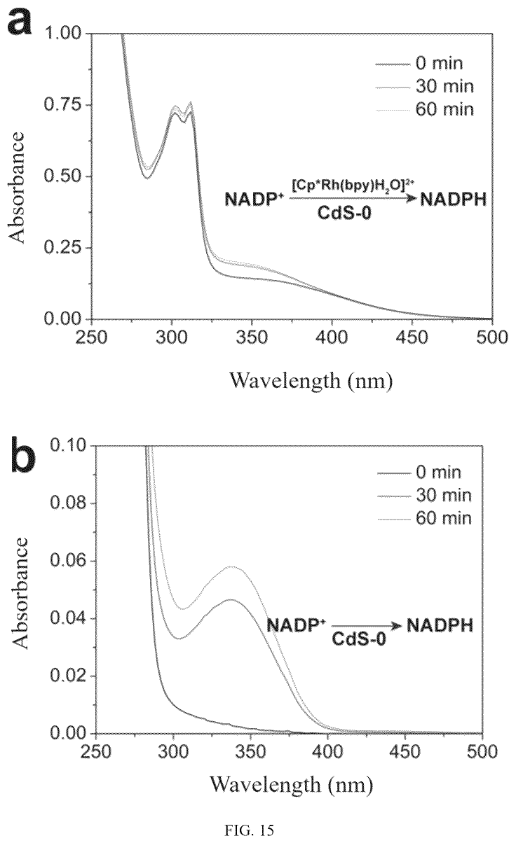

The UV-Vis absorption spectra of the reaction solutions provided in Example S14 and Example DS3 were measured. The results are shown in FIG. 15.

Part a of FIG. 15 presents the UV-Vis absorption spectrum of the reaction solution from Example DS3, while part b of FIG. 15 presents the UV-Vis absorption spectrum of the reaction solution from Example S14. As can be seen from the figures, the regeneration of the coenzyme NADPH can be achieved during the photocatalytic process, regardless of the presence or absence of an electron mediator. As evident from part a of FIG. 15, when an electron mediator was utilized, the absorption intensity of the coenzyme NADPH increased by 0.0544 over the period from 0 min to 60 mins. As evident from part b of FIG. 15, in the absence of an electron mediator, the absorption intensity of the coenzyme NADPH increased by 0.0542 over the same time interval.

By combining the observations from FIGS. 14-15, it is clear that, regardless of the presence or absence of an electron mediator, the CdS photocatalyst provided in Example 4 exhibits higher photocatalytic activity for coenzyme regeneration than the CdS photocatalyst provided in Example 1. This enhanced activity is attributed to the CdS material provided in Example 4 having higher photogenerated charge separation efficiency and a faster photogenerated carrier migration rate.

### (7) Photocatalytic Coenzyme Regeneration Mechanism

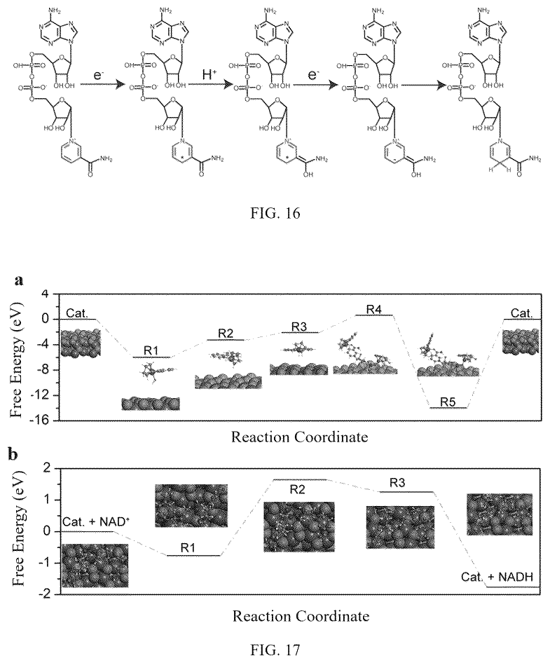

For photocatalytic systems lacking an electron mediator, the pathway for NADH coenzyme generation is illustrated in FIG. 16.

As is clearly evident from FIG. 16, within this photocatalytic system, an NAD⁺ molecule accepts a photogenerated electron from CdS to form an NAD⁺ radical (NAD⋅). Subsequently, the NAD⋅ accepts a proton, leading to the formation of an enol-NADH radical cation (enol-NADH⋅⁺). From a thermodynamic perspective, the enol radical cation is favored over its corresponding keto form, as the energy required to remove an electron from the π-orbital of the enol is lower than that required to remove an electron from the n-orbital of the keto form. Upon accepting a second electron, the enol-NADH⋅+ forms enol-NADH. Finally, the enol-NADH undergoes an intramolecular rearrangement to yield 1,4-NADH.

Density Functional Theory (DFT) calculations were employed to determine the potential energy profiles for the metal sulfide-catalyzed regeneration of NADH. Specifically, under conditions utilizing an electron mediator ([Cp*Rh(bpy)H₂O]²⁺), the potential energy profile for NADH regeneration catalyzed by the metal sulfide prepared in Example 4 is depicted in part a of FIG. 17; conversely, under conditions where no electron mediator ([Cp*Rh(bpy)H₂O]²⁺) was added, the potential energy profile for NADH regeneration catalyzed by the same metal sulfide is depicted in part b of FIG. 17.

As shown in part a of FIG. 17, the adsorption of the electron mediator [Cp*Rh(bpy)H₂O]²⁺ onto the surface of the metal sulfide CdS is a spontaneous process, with an adsorption energy of −5.979 eV. When the CdS is photoexcited, photogenerated electrons and photogenerated holes are produced; the holes oxidize triethanolamine to generate H⁺, while the electrons react with [Cp*Rh(bpy)H₂O]²⁺ to form [Cp*Rh(bpy)]. The formation of [Cp*Rh(bpy)] is a non-spontaneous process that requires overcoming an energy barrier of 2.722 eV. Subsequently, [Cp*Rh(bpy)] accepts a proton (H⁺) to form [Cp*Rh(bpy)H]⁺; this is an endothermic process, absorbing 1.173 eV of energy. The interaction between [Cp*Rh(bpy)H]⁺ and NAD⁺ is thermodynamically non-spontaneous, requiring the system to surmount an energy barrier of 2.732 eV. NAD⁺ coordinates to the Cp*Rh metal center via its amide oxygen atom. It is worth noting that the subsequent reduction reaction, following the interaction of [Cp*Rh(bpy)H]⁺ with NAD⁺ to form the transition state [Cp*Rh(bpy)H-NAD]²⁺, is a spontaneous process, releasing 14.621 eV of energy. This process achieves the reduction of NAD⁺, yielding NADH. Finally, under the influence of H2O, 1,4-NADH desorbs, and [Cp*Rh(bpy)H₂O]²⁺ is regenerated.

As illustrated in part b of FIG. 17, for catalytic systems devoid of electron mediators, the interaction between NAD⁺ and photo-excited CdS, generating an electron to form the radical species NAD⋅, is a spontaneous process, releasing 0.765 eV of energy. Subsequently, the process of NAD⋅ accepting a proton to form enol-NADH⋅⁺ requires surmounting an energy barrier of 2.406 eV; when enol-NADH⋅⁺ accepts an additional electron to form enol-NADH, this constitutes a spontaneous process, releasing 0.383 eV of energy. Finally, enol-NADH undergoes intramolecular rearrangement to form 1,4-NADH, which is also a spontaneous process, as the formation of the more stable 1,4-NADH product lowers the free energy of the system.

The foregoing descriptions serve merely as optional embodiments of the present application and are not intended to limit its scope. For those skilled in the art, the present application is susceptible to various modifications and variations. Any modifications, equivalent substitutions, improvements, or the like, provided they fall within the spirit and principles of the present application shall be deemed to be encompassed within the scope of the claims appended hereto.

What is claimed is:

1. A coenzyme regeneration system, comprising: a metal sulfide, an oxidized coenzyme, and a solvent; wherein the metal sulfide has a hexagonal phase crystal structure.

2. The coenzyme regeneration system according to claim 1, wherein the metal sulfide comprises at least one of cadmium sulfide, tin sulfide, zinc sulfide, indium sulfide, and molybdenum sulfide.

3. The coenzyme regeneration system according to claim 2, wherein the metal sulfide is cadmium sulfide, and the cadmium sulfide has a feather-like morphology.

4. The coenzyme regeneration system according to claim 2, wherein the metal sulfide is cadmium sulfide, and a lattice spacing of a (100) crystal plane of the cadmium sulfide is 0.358 nm.

5. The coenzyme regeneration system according to claim 2, wherein the metal sulfide is cadmium sulfide, and an X-ray photoelectron spectrum of the cadmium sulfide exhibits characteristic peaks within ranges of 160 eV to 162 eV, 162 eV to 164 eV, 404 eV to 407 eV, and 410 eV to 414 eV.

6. The coenzyme regeneration system according to claim 2, wherein the metal sulfide is cadmium sulfide, and the cadmium sulfide comprises cadmium sulfide single crystals.

7. The coenzyme regeneration system according to claim 2, wherein the metal sulfide is tin sulfide, and the tin sulfide comprises tin sulfide nanosheets, wherein the tin sulfide nanosheets have a thickness of 2 nm to 50 nm and a lateral dimension of 50 nm to 10 μm.

8. The coenzyme regeneration system according to claim 1, wherein within the coenzyme regeneration system, the oxidized coenzyme has an initial concentration of 0.1 mM to 1 mM, and the metal sulfide has an initial concentration of 1.25 mg/mL to 25 mg/mL.

9. The coenzyme regeneration system according to claim 1, wherein the oxidized coenzyme comprises at least one of NAD⁺ and NADP⁺.

10. The coenzyme regeneration system according to claim 1, wherein the solvent comprises at least one of triethanolamine and deionized water.

11. The coenzyme regeneration system according to claim 1, wherein the coenzyme regeneration system further comprises a pH buffering agent, and the pH buffering agent comprises a phosphate-based buffering agent.

12. The coenzyme regeneration system according to claim 1, wherein the coenzyme regeneration system has a pH value of 7 to 8.5.

13. A coenzyme regeneration method, comprising the following step:
subjecting the coenzyme regeneration system according to claim 1 to a photocatalytic reaction under irradiation of a visible light, thereby converting the oxidized coenzyme within the coenzyme regeneration system into a reduced coenzyme.

14. The coenzyme regeneration method according to claim 13, wherein the visible light has a wavelength of 400 nm to 720 nm.

15. The coenzyme regeneration method according to claim 13, wherein a vertical distance between the visible light source and the coenzyme regeneration system is 5 cm to 30 cm.

16. The coenzyme regeneration method according to claim 13, wherein the visible light source has a power of 50 W to 450 W.

17. The coenzyme regeneration method according to claim 13, wherein a duration of the photocatalytic reaction is 10 mins to 100 mins.

18. The coenzyme regeneration method according to claim 13, wherein the reduced coenzyme comprises at least one of 1,4-NADH and NADPH.

19. The coenzyme regeneration method according to claim 13, wherein the metal sulfide comprises at least one of cadmium sulfide, tin sulfide, zinc sulfide, indium sulfide, and molybdenum sulfide.

20. The coenzyme regeneration method according to claim 13, wherein the metal sulfide is cadmium sulfide, and the cadmium sulfide has a feather-like morphology.
